(19) United States
(12) Patent Application Publication
(10) Pub. No.: US 2011/0086030 A1
(43) Pub. Date: Apr. 14, 2011

(54) INHIBITION OF CANCER METASTASIS

(75) Inventor: Jerry Ware, Little Rock, AR (US)

(73) Assignee: BOARD OF TRUSTEES OF THE UNIVERSITY OF ARKANSAS, Little Rock, AR (US)

(21) Appl. No.: 12/967,272

(22) Filed: Dec. 14, 2010

Related U.S. Application Data
(62) Division of application No. 11/952,198, filed on Dec. 7, 2007.
(60) Provisional application No. 60/868,965, filed on Dec. 7, 2006.

(51) Int. Cl. A61K 39/395; A61P 35/00

(52) U.S. Cl. 424/135.1; 424/130.1; 424/141.1; 424/133.1

(57) ABSTRACT

The present invention provides methods for inhibiting tumor cell metastasis. In particular, the invention provides methods for reducing tumor cell malignancy by administering to a subject an antibody that inhibits glycoprotein Ibα, such that tumor cell malignancy is reduced.

### CROSS-REFERENCE TO RELATED APPLICATIONS

This application is a divisional application of U.S. patent application Ser. No. 11/952,198, filed Dec. 7, 2007, which claims priority to U.S. Provisional Application Ser. No. 60/868,965 filed on Dec. 7, 2006, each of which is hereby incorporated by reference in its entirety.

### GOVERNMENT SUPPORT

The present invention was made with government support under HL50545 and CA095458 from the National Institutes of Health. The United States Government has certain rights in this invention.

### FIELD OF THE INVENTION

The present invention relates to methods for inhibiting the metastasis of malignant tumor cells.

### BACKGROUND OF THE INVENTION

Malignant tumors (cancers) are the second leading cause of death, after heart disease, among adults in the United States and other industrialized countries. Cancer is characterized by the uncontrolled proliferation of abnormal cells in a tissue or organ, resulting in a neoplasm. The neoplasm may form a solid mass or tumor. Cancer is also characterized by the invasion of the tumor cells into adjacent tissues, and the spread (metastasis) of tumor cells via the bloodstream or lymphatic system to other parts of the body.

Tumor metastasis involves detachment of malignant tumor cells from the primary tumor, escape through the surrounding extracellular matrix, intravasation into microvessels, extravasation from microvessels, and proliferation in a foreign environment to form a secondary or metastatic tumor. Once a cancer has metastasized, the prognosis is poor. Most cancer treatment regimes focus on eradicating the malignant tumor cells through surgery, irradiation, and/or chemotherapy. Most chemotherapeutic agents target rapidly dividing cells or abnormal cells with disregulated proteins. There is a great need, therefore, for compositions and methods that effectively treat or suppress tumor cell malignancy and/or metastasis.

### SUMMARY OF THE INVENTION

Among the various aspects of the present invention, therefore, is the provision of a method for reducing tumor cell metastasis in a subject. The method comprises administering to the subject an antibody that inhibits glycoprotein Ibα such that tumor cell metastasis is reduced.

Another aspect of the invention encompasses a method for reducing tumor cell malignancy in a subject. The method comprises administering to the subject an antibody that inhibits glycoprotein Ibα such that tumor cell malignancy is reduced.

A further aspect of the invention provides a method for inhibiting formation of a tumor cell embolism in a subject. The method comprises administering to the subject an antibody that inhibits glycoprotein Ibα such that formation of the tumor cell embolism is inhibited.

Other aspects and features of the invention will be in part apparent and in part pointed out hereinafter.

### DESCRIPTION OF THE FIGURES

FIG. 1 presents schematics of variant platelet GP Ib-IX receptor complexes expressed on the surface of circulating platelets. (a) The WT GP Ib-IX complex consists of three distinct gene products. The disulfide-linked α- and β-subunits of GP Ib and the noncovalently associated GP IX. (b) A mouse model of GP Ib-IX deficiency (GP1b^{-/-}) lacks the gene encoding GP Ibα, resulting in a missing complex owing to the three-subunit requirement for efficient surface expression of the complex. (c) A variant GP Ib-IX deficiency (IL-4R) in which an extracellular domain from the interleukin-4 receptor is fused to a few residues from the GP Ibα extracellular domain and the complete GP Ibα transmembrane and cytoplasmic domains. (d) A rescue of mouse GP Ibα deficiency was performed by transgenic expression of the human GP Ibα subunit (hTg^{WT}). (e) A rescue of mouse GP Ibα deficiency was performed by transgenic expression of the human GP Ibα subunit lacking the six terminal residues (hTg^{Y605X}).

FIG. 2 illustrates the metastatic tumor foci observed in wild-type mice and treated mice after injection of B16-F10.1 melanoma cells (1×10⁵). Fourteen days later, the lungs were removed, and surface-visible tumors were counted in normal (WT), GP Ib-IX deficient mice (GP1b^{-/-}), and mice with an absent extracellular domain of platelet GP Ibα (IL-4R). (a) Box plot data represent range, median, and quartile values. Median values are represented by the horizontal line. P values comparing each group are shown. (b) Two representative metastatic lungs are shown for comparison.

FIG. 3 presents flow cytometry profiles of B16F10.1 cells mixed with washed platelets at a ratio of 1:200 (B16:platelets). Tumor cell forward scatter profile (upper) was analyzed for fluorescence (lower) produced by a platelet-specific phycoerythrin (PE)-labeled rat anti-mouse CD41 (αIIb, glycoprotein IIb) monoclonal antibody. Fluorescent profiles of tumor cells in the presence of platelets from normal mice (C57BL/6J, black line), GP1b^{-/-} animals (dark gray), and IL-4R animals (light gray) are shown. Labeled B16 cells in the absence of washed platelets are shown for comparison (shaded gray area).

FIG. 4 illustrates an in vivo model of thrombus formation. Blood flow through the carotid is presented for GP Ib-IX deficient mice expressing the normal human GP Ibα subunit (hTg^{WT}) (left tracings) and GP Ib-IX deficient mice expressing a truncated human GP Ibα subunit lacking the carboxyl-terminal 6 aa (hTg^{Y605X}) (right tracings). A 10% FeCl₃-soaked filter was placed on the surface of an exposed carotid artery for 3 min, and blood flow was measured with a laser Doppler probe after removal of the filter (indicated by the arrow). The representative tracings from three different mice from each colony follow blood flow from a maximum value (top of the graph) to a minimum value that represents occlusion of the carotid (bottom of the graph). The graphs are representative of 10 individual measurements from each mouse strain.

FIG. 5 illustrates metastatic foci in GP Ib-IX deficient mice expressing a human GP Ibα subunit (hTg^{WT}) and GP Ib-IX deficient mice with a truncated cytoplasmic tail of GP Ibα (hTg^{Y605X}B16-F10.1 melanoma cells (1×10⁵) were injected via a mouse tail vein, the lungs were removed fourteen days later, and surface-visible tumors were counted. Box plot data represent range, median, and quartile values. Median values are represented by the horizontal lines.

### DETAILED DESCRIPTION

A method for inhibiting tumor cell metastasis has been discovered. The adhesive properties of circulating blood platelets have long been recognized as critical in blood clotting and thrombosis, but it appears that they are also involved in metastasis and tumorigenesis. Interactions between tumor cells and platelets in a microvessel appear to contribute to the lodgement of tumor cells in a microvessel or the attachment of tumor cells to the microvessel wall, which is an important step in metastasis. Once tumor cells are immobilized in a microvessel, they may extravasate the microvessel, invade the surrounding tissue, and proliferate to form a metastatic tumor.

The platelet-specific adhesion receptor, glycoprotein Ib-IX (GP Ib-IX), plays a major role in platelet aggregation and elaboration of a fibrin-rich network produced by coagulation factors. GP Ib-IX comprises two non-covalently associated integral membrane proteins, glycoprotein Ib (comprising an alpha subunit and a beta subunit) and glycoprotein IX. The adhesive ligand of GP Ib-IX is von Willebrand factor (vWF), a constituent of the blood plasma and the subendothelial matrix. The vWF binding site of the GP Ib-IX complex is located in the extracellular domain of the GP Ibα subunit.

### I. Method for Inhibiting Tumor Cell Metastasis

One aspect of the invention encompasses a method for inhibiting tumor cell metastasis in a subject. The method comprises administering an inhibitor of GP Ibα to the subject, whereby tumor cell metastasis in the subject is inhibited. Inhibition of tumor cell metastasis may be manifested by a reduction in the number or distribution of metastatic tumors in the treated subject relative to an untreated subject. In one embodiment, the number of metastatic tumors may be reduced two-fold. In another embodiment, the number of metastatic tumors may be reduced ten-fold. In still another embodiment, the number of metastatic tumors may be reduced 50-fold. In yet another embodiment, the number of metastatic tumors may be reduced 200-fold. In a further embodiment, the number of metastatic tumors may be reduced to such an extent such that no metastatic tumors are detectable. In still another embodiment, metastatic tumors may be restricted to one organ or tissue, rather than being spread to two or more organs or tissues

The method comprises administering an inhibitor of GP Ibα to the subject. Inhibition of GP Ibα or complexes comprising GP Ibα may prevent accumulation of platelets and formation of the fibrin-rich network around the tumor cells, such that the tumor cells are not immobilized in the microvessel and may not form a metastatic tumor. Lack of functional GP Ibα (and consequently, GP Ib-IX), as demonstrated in the examples, reduces the number of metastatic foci.

(a) GP Ibα Inhibitors

The GP Ibα inhibitor may be a peptide, an antibody, a small molecule, or a venom protein. In general, the GP Ibα inhibitor perturbs interactions between tumor cells, platelets, endothelial cells, and coagulation factors such that the tumor cells are prevented from attaching to the surface of a microvessel, but normal hemostasis is not affected.

i. Peptide Inhibitors

The GP Ibα inhibitor may be a peptide. In some embodiments, the peptide may correspond to a region of the GP Ibα binding domain of vWF but lack the bridging activity of mature vWF such that it is unable to form a multimeric complex. In general, the GP Ibα binding domain of vWF comprises a region from about amino acid residue 431 to about amino acid residue 750 of mature vWF. Thus, the peptide inhibitor of GP Ibα may be a peptide that consists of amino acid residues 431-750 (SEQ ID NO:1) of mature vWF or a fragment thereof. Exemplary GP Ibα inhibitors that correspond to a region of vWF include peptides that consist of amino acid residues 441-709 (SEQ ID NO:2) of mature vWF, amino acid residues 441-704 (SEQ ID NO:3) of mature vWF, amino acid residues 441-700 (SEQ ID NO:4) of mature vWF, amino acid residues 441-696 (SEQ ID NO:5) of mature vWF, amino acid residues 475-733 (SEQ ID NO:6) of mature vWF, amino acid residues 492-733 (SEQ ID NO:7) of mature vWF, amino acid residues 508-733 (SEQ ID NO:8) of mature vWF, amino acid residues 508-709 (SEQ ID NO:9) of mature vWF, amino acid residues 508-704 (SEQ ID NO:10) of mature vWF, and amino acid residues 508-700 (SEQ ID NO:11) of mature vWF, all of which are detailed in U.S. Pat. No. 5,900,476, which is hereby incorporated in its entirety by reference.

A skilled practitioner will recognize that peptides may be substantially similar to the peptides described above in that an amino acid residue may be substituted with another amino acid residue having a similar side chain without affecting the function of the peptide. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acid substitution groups include: valine-leucine-isoleucine, phenylalanine-tyrosine, lysine-arginine, alanine-valine, and asparagine-glutamine. Thus, the peptide inhibitor may have one or more conservative amino acid substitutions. In one embodiment the peptide may have an amino acid sequence at least 90% identical to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11, and the peptide retains function. That is, the peptide functions as an inhibitor of GP Ibα and the peptide is unable to form a multimer complex. In another embodiment, the peptide may have an amino acid sequence at least 95% identical to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11, and the peptide retains function. In a further embodiment, the peptide may have an amino acid sequence at least 97% identical to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11, and the peptide retains function. In yet another embodiment, the peptide may have an amino acid sequence at least 99% identical to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11, and the peptide retains function.

The degree of sequence identity between two amino acid sequences may be determined using the BLASTp algorithm of Karlin and Altschul (Proc. Natl. Acad. Sci. USA 87:2264-2268, 1993). The percentage of sequence identity is determined by comparing two optimally aligned sequences over a comparison window, wherein the portion of the amino acid sequence in the comparison window may comprise additions or deletions (i.e., gaps) as compared to the reference sequence (which does not comprise additions or deletions) for optimal alignment of the two sequences. The percentage is calculated by determining the number of positions at which an identical amino acid occurs in both sequences to yield the number of matched positions, dividing the number of matched positions by the total number of positions in the window of comparison and multiplying the result by 100 to yield the percentage of sequence identity.

The vWF-related peptide inhibitor of GP Ibα may be a fragment of vWF generated by digestion with trypsin or another endopeptidase. The fragment or fragments of digested vWF may be purified and isolated using techniques that are well known in the art. Alternatively, the vWF-related peptide inhibitor of GP Ibα may be recombinantly produced from DNA encoding sequences using molecular biology techniques well know to those with skill in the art. The recombinant peptide may be produced in bacterial cell, eukaryotic cells, or mammalian cells. The vWF-related peptide inhibitor of GP Ibα may also be synthesized in vitro using solid phase synthesis techniques that are well known in the art. Guidance for any of the above-mentioned techniques may be found in reference texts such as Current Protocols in Molecular Biology (Ausubel et al., John Wiley & Sons, New York, 2003) or Molecular Cloning: A Laboratory Manual (Sambrook & Russell, Cold Spring Harbor Press, Cold Spring Harbor, N.Y., 3rd edition, 2001).

Furthermore, the vWF-related peptide inhibitor of GP Ibα lacks the bridging activity of mature vWF and is unable to form a multimeric complex. Typically, mature vWF monomers arrange into large multimeric complexes via disulfide bonds, glycosylation reactions, and other interactions. Thus, although the vWF-related peptide inhibitor of GP Ibα may comprise cysteine residues and may have intrapeptide disulfide bonds, it may not form interpeptide disulfide bonds. Accordingly, the cysteine residues in the vWF-related peptide inhibitor of GP Ibα may be substituted with other amino acids via PCR-based site directed mutagenesis, for example. Alternatively, the cysteine residues may be reduced (e.g., with β-mercaptoethanol or dithiothreitol), or the cysteine residues may be reduced and permanently alkylated (e.g., by reaction with iodoacetamide). Additionally, the vWF-related peptide may not be glycosylated, and it may not interact non-covalently with other vWF-derived peptides to form a multimeric complex.

In other embodiments, the peptide that inhibits GP Ibα may be a membrane-permeable peptide corresponding to a region of the intracellular domain of GP Ibα. In general, the intracellular domain of GP Ibα interacts with cytoplasmic proteins to mediate downstream signaling events. For example, the membrane-permeable peptide inhibitor of GP Ibα may be a myristoylated phospho-peptide that corresponds to the binding site of the 14-3-3ζ protein or another protein in the intracellular C-terminal region of GP Ibα. A myristoylated phospho-peptide that corresponds to amino acid residues 602-610 of GP Ibα inhibits the binding of vWF to platelets and inhibits vWF-mediated platelet adhesion (Du et al., 2005, Blood 106(6):1975-1981, which is incorporated in its entirety by reference). Membrane-permeable peptides that correspond to other regions of the intracellular domain of GP Ibα may also be used to inhibit the activity of GP Ibα. As an example, a peptide corresponding to amino acid residues 557-569 of GP Ibα linked to a nine-arginine permeating tag inhibits interactions between GP Ib and vWF (David et al., 2006, J. Thromb. Haemost. 4(12):2645-2655, which is herein incorporated by reference in its entirety). Peptides that correspond to the intracellular domain of GP Ibα may be obtained using any of the techniques described above.

ii. Antibody Inhibitors

In other embodiments, the GP Ibα inhibitor may be an antibody or a fragment thereof. In general, the antibody or a fragment thereof will inhibit the activity of GP Ibα with regard to metastasis, but will not perturb normal hemostasis. In some embodiments, the antibody that inhibits GP Ibα may be a single chain antibody. The single chain antibody may be a single chain Fv (scFv) fragment in which the variable regions of the light and heavy chains are joined by a flexible linker moiety. The single chain Fv antibody may be generated using methods disclosed in U.S. Pat. No. 4,946,778 or using phage display library techniques (Huse et al., 1989, Science 246:1275-1281; McCafferty et al., 1990, Nature 348:552-554) (each of these is incorporated in its entirety by reference). In other embodiments, the antibody that inhibits GP Ibα may be an antibody fragment. Suitable antibody fragments include Fab fragments, Fab' fragments, Fd fragments (i.e., heavy chain variable domain), and Fv fragments. These antibody fragments may be generated by enzymatic cleavage, via recombinant libraries, expression libraries, phage display techniques, or other means known to those of skill in the art (for additional guidance, see e.g., Coico, R. (ed), Current Protocols in Immunology, 2007, John Wiley & Sons, Inc., New York). In yet other embodiments, the antibody that that inhibits GP Ibα may be a camelid antibody, which is a small antibody molecule that lacks light chains (Hamers-Casterman et al., 1993, Nature 363(6428):446-448). In further embodiments, the antibody that inhibits GP Ibα may be a chimeric antibody or antibody fragment. Alternatively, the antibody that inhibits GP Ibα may be a humanized antibody or antibody fragment. Those of skill in the art are familiar with techniques to generate chimeric or humanized antibodies.

In some embodiments, the antibody that inhibits GP Ibα may bind to the extracellular domain of GP Ibα. Monoclonal antibodies against GP Ibα have been described (Kanaji et al., 2003, J. Biol. Chem. 278(41):39452-60; Federici et al., 2004, Haematologica 89(1):77-85; Berndt et al., 1985, Eur. J. Biochem. 151:637-649; Ruan et al., 1987 Blood 69:570-577), each of which is hereby incorporated by reference in its entirety. Monoclonal antibodies against GP Ibα are also available commercially (e.g., from R+D Systems Inc., Minneapolis, Minn.). In other embodiments, the antibody that inhibits GP Ibα may bind to the GP Ibα binding domain of vWF. Monoclonal antibodies

PATENT against this domain have been generated (Kageyama et al., 1997, Br. J. Pharmacol. 122(1):165-171; Celikel et al., 1997 Blood Cells Mol. Dis. 23(1):123-134; U.S. Pat. Publ. No. 2005/0136056), as well as a humanized monoclonal antibody (AJW200) against vWF (Kageyama et al., 2002, Arterioscler. Thromb. Vasc. Biol. 22(1):187-192), each of which is incorporated by reference in its entirety.

iii. Small Molecule Inhibitors and Venom Protein Inhibitors

In still other embodiments, the GP Ibα inhibitor may be a small molecule such as aurintricarboxylic acid (ATA) (Phillips et al., 1988, Blood 6:1898-1903) or a snake venom protein that is a GP Ibα inhibitor. Suitable venom proteins include agkicetin-C (Chen et al., 2000, Thromb. Haemost. 83:119-126), agkisten (Yeh et al., 2001, Br. J. Pharmacol. 132(4):843-850), agkistrodon (Li et al., 2005, Biochem. Biophys. Res. Commun. 332(3):904-912), CHH-B (Andrew et al., 1996, Biochem. 35:12629-12639), crotalin (Chang et al., 1998, Blood 91(5):1582-1589), echicetin (Polgar et al., 1997, Biochem. J. 323:533-537), flavocetin-A (Kukuda et al, 2000, Biochem. 39:1915-1923), jararaca GPIb-BP (Fujimura et al., 1995, Thromb. Haemost. 74(2):743-750), mamushigin (Sakurai et al., 1998, Thromb. Haemost. 79:1199-1207), tokaracetin (Kawasaki et al., 1995, Biochem. J. 308(3):947-953), each of which is incorporated by reference in its entirety. The venom protein may be purified from venom using conventional techniques. Alternatively, the venom protein may be produced using recombinant DNA technologies well known to those of skill in the art. It is envisioned that a fragment of a venom protein or a derivative of venom protein may also be used in the method of the invention.

(b) Administration

The GP Ibα inhibitor may be administered to the subject in accord with known methods, such as intravenous administration as a bolus or by continuous infusion over a period of time, by intramuscular, intraperitoneal, subcutaneous, intra-articular, intrasynovial, intrathecal, oral, topical, or inhalation routes. Optionally, administration may be performed through mini-pump infusion using various commercially available devices.

Agents administered parenterally, i.e., intravenously, intramuscularly, etc., may include a sterile diluent such as water, saline solution, a pharmaceutically acceptable polyol such as glycerol, propylene glycol, polyethylene glycols, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the solution may be adjusted with acids or bases such as hydrochloric acid or sodium hydroxide. Preparations for oral administration generally include an inert diluent or an edible carrier. They may be include a pharmaceutically compatible binding agent such as microcrystalline cellulose, gum tragacanth or gelatin; an excipient such as starch or lactose; a disintegrating agent such as alginic acid, Primogel, or corn starch; a lubricant such as magnesium stearate or sterotes; a glidant such as colloidal silicon dioxide; a sweetening agent such as sucrose or saccharin; and/or a flavoring agent such as peppermint, methyl salicylate, or citrus flavoring. Oral preparations may be enclosed in gelatin capsules, compressed into tablets, or prepared as a fluid carrier. For administration by inhalation, the agent is generally delivered in the form of an aerosol spray from a pressurized container or dispenser that contains a suitable propellant, e.g., a gas such as carbon dioxide, or a nebulizer. For topical (e.g., transdermal or transmucosal) administration, penetrants appropriate to the barrier to be permeated are generally included in the preparation. Transmucosal administration may be accomplished through the use of nasal sprays or suppositories, and transdermal administration may be via ointments, salves, gels, patches, or creams as generally known in the art.

The amount of the GP Ibα inhibitor that is administered to the subject can and will vary depending upon the type of inhibitor, the subject, and the particular mode of administration. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The subject administered the GP Ibα inhibitor can and will vary. Suitable subjects include animals and humans. The animal may be a companion animal such as a cat or a dog; a research animal such as a mouse, a rat, or a rabbit; an agricultural animal such as a cow, a pig, a horse, a goat, or a sheep; a zoo animal; or a primate such as a chimpanzee, a monkey, or a gorilla. In preferred embodiments, the subject is a human.

(c) Tumor Cell

Most malignant tumors and other neoplasms have the capacity to metastasize and form secondary or metastatic tumors in other locations in the body. Metastatic tumors are common in the late stages of cancer. Cancers that may metastasize include carcinoma, lymphoma, blastoma, sarcoma, and leukemia or lymphoid malignancies. Notable exceptions include glioma, basal cell carcinoma, and squamous cell carcinoma, which typically do not metastasize. More specific examples of cancers that frequently metastasize include lung cancer (i.e., small-cell lung cancer, non-small cell lung cancer, adenocarcinoma and squamous carcinoma of the lung), breast cancer, melanoma, colon cancer, kidney cancer, prostate cancer, and pancreatic cancer. Cancers that may metastasize also include gastric or stomach cancer including gastrointestinal cancer, cervical cancer, ovarian cancer, endometrial or uterine carcinoma, vulval cancer, liver cancer, bladder cancer, cancer of the urinary tract, cancer of the peritoneum, salivary gland carcinoma, thyroid cancer, anal carcinoma, penile carcinoma, testicular cancer, bone cancer, brain cancer, head and neck cancer, multiple myeloma, and B-cell lymphoma.

II. Method for Reducing Tumor Malignancy

Another aspect of the invention is a method for reducing tumor malignancy in a subject. In general, tumor malignancy is characterized by aggressive cell growth and proliferation, the ability to invade adjacent tissues, and the ability to metastasize or spread to distant sites. Thus, the tumor malignancy may be reduced by reducing or eliminating the proliferation of tumor cells, reducing or eliminating the invasiveness of tumor cells, and/or reducing or eliminating the metastasis of tumor cell.

The method of the invention comprises administering a GP Ibα inhibitor to the subject. Inhibition of GP Ibα or complexes comprising GP Ibα may prevent platelet accumulation and formation of a fibrin-rich matrix around the displaced primary tumor cells, such that tumor cells may not adhere to the wall of a microvessel. If the tumor cells do not adhere to the wall of the microvessel, they may not be able to escape from the microvessel, invade the surrounding tissue, and proliferate to form a secondary or metastatic tumor. Thus, tumor cell malignancy may be reduced by reducing or eliminating tumor cell metastasis in the treated subject relative to an untreated subject.

The GP Ibα inhibitor may be a peptide, an antibody, a small molecule, or a venom protein, as detailed above in section I(a). Modes of administration of the GP Ibα inhibitor were detailed above in section I(b). The different types of cancer whose malignancy may be reduced were detailed above in section I(c).

The method for reducing tumor malignancy may further comprise administering an additional treatment in addition to the GP Ibα inhibitor. In general, the additional treatment is chosen to treat or eliminate the primary tumor or neoplasm. Accordingly, the additional treatment may remove or destroy the tumor cells, reduce the proliferation of the tumor cells, reduce or eliminate angiogenesis in the tumor such that the size of the tumor is restricted, and/or reduce the invasiveness of the tumor cells. Any of these processes, therefore, may further reduce the malignancy of the tumor. The additional treatment may be administered prior to, concurrent with, or after administration of the GP Ibα inhibitor. The additional treatment may include surgery, radiation therapy, chemotherapy, or a combination thereof.

Depending upon the type of cancer, a surgical procedure may entail removal of the tumor only, removal of the entire organ, and/or removal of regional lymph nodes. Radiation therapy refers to the use of ionizing radiation to kill cancer cells and shrink tumors. Radiation therapy (or irradiation) may be administered externally via external beam radiotherapy (EBRT) or internally via brachytherapy. Radiation therapy may be used to treat almost every type of solid tumor, including cancers of the brain, breast, cervix, larynx, lung, pancreas, prostate, skin, stomach, uterus, or soft tissue sarcomas. Radiation may also be used to treat leukemia and lymphoma. Radiation dose to each site depends on a number of factors, including the radiosensitivity of each cancer type and whether there are tissues and organs nearby that may be damaged by radiation.

Chemotherapeutic agent refers to a chemical compound that is useful in the treatment of cancer. The compound may be a cytotoxic agent that affects rapidly dividing cells in general, or it may be a targeted therapeutic agent that affects the deregulated proteins of cancer cells. The chemotherapeutic agent may be an alkylating agent, an anti-metabolite, an anti-tumor antibiotic, an anti-cytoskeletal agent, a topoisomerase inhibitor, an anti-hormonal agent, a targeted therapeutic agent, or a combination thereof. Non-limiting examples of alkylating agents include altretamine, benzodopa, busulfan, carboplatin, carboquone, carmustine, chlorambucil, chlornaphazine, cholophosphamide, chlorozotocin, cisplatin, cyclosphosphamide, dacarbazine (DTIC), estramustine, fotemustine, ifosfamide, improsulfan, lomustine, mechlorethamine, mechlorethamine oxide hydrochloride, melphalan, meturedopa, nimustine, novembichin, phenesterine, piposulfan, prednimustine, ranimustine; temozolomide, thiotepa, triethylenemelamine, trietylenephosphoramide, triethylenethiophosphaoramide, trimethylolomelamine, trofosfamide, uracil mustard and uredopa. Suitable anti-metabolites include, but are not limited to aminopterin, ancitabine, azacitidine, 6-azauridine, capecitabine, carmofur, cytarabine or cytosine arabinoside (Ara-C), dideoxyuridine, denopterin, doxifluridine, enocitabine, floxuridine, fludarabine, 5-fluorouracil (5-FU), gemcetabine, leucovorin (folinic acid), 6-mercaptopurine, methotrexate, pemetrexed, pteropterin, thiamiprine, trimetrexate, and thioguanine. Non-limiting examples of suitable anti-tumor antibiotics include aclacinomysin, actinomycin, adriamycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin. Non-limiting examples of suitable anti-cytoskeletal agents include colchicines, docetaxel, macromycin, paclitaxel (taxol), vinblastine, vincristine, vindesine, and vinorelbine. Suitable topoisomerase inhibitors include, but are not limited to, amsacrine, etoposide (VP-16), irinotecan, RFS 2000, teniposide, and topotecan. Non-limiting examples of suitable anti-hormonal agents such as aminoglutethimide, aromatase inhibiting 4(5)-imidazoles, bicalutamide, finasteride, flutamide, goserelin, 4-hydroxytamoxifen, keoxifene, leuprolide, LY117018, mitotane, nilutamide, onapristone, raloxifene, tamoxifen, toremifene, and trilostane. No-limiting examples of targeted therapeutic agents include a monoclonal antibody such as alemtuzumab, bevacizumab, capecitabine, cetuximab, gemtuzumab, heregulin, rituximab, trastuzumab; a tyrosine kinase inhibitor such as imatinib mesylate; and a growth inhibitory polypeptide such as erythropoietin, interleukins (e.g., IL-1, IL-2, IL-3, IL-6), leukemia inhibitory factor, interferons, thrombopoietin, TNF-α, CD30 ligand, 4-1BB ligand, and Apo-1 ligand. Also included are pharmaceutically acceptable salts, acids, or derivatives of any of the above listed agents. The mode of administration of the chemotherapeutic agent can and will vary depending upon the agent and the type of tumor or neoplasm. Suitable modes of administration were detailed above in section I(b). A skilled practitioner will be able to determine the appropriate dose of the chemotherapeutic agent.

III. Method for Inhibiting Formation of a Tumor Cell Embolism

A further aspect of the invention encompasses a method for inhibiting formation of a tumor cell embolism in a subject. The method comprises administering a GP Ibα inhibitor to the subject, whereby formation of a tumor cell embolism in a microvessel is inhibited relative to an untreated subject. The inhibition of tumor cell embolism formation may be partial or it may be essentially complete. For example, the method may inhibit the formation of tumor cell emboli by at least 20%, at least 50%, at least 70%, at least 90%, at least 95%, at least 99%, or at least 99.99%

The method comprises administering a GP Ibα inhibitor to the subject. Inhibition of GP Ibα or complexes comprising GP Ibα may prevent platelet accumulation and formation of a fibrin-rich matrix around the displaced primary tumor cells such that tumor cells may not adhere to the wall of a microvessel and form a tumor cell embolus. If a tumor cell embolus does not form, the tumor cells may not be able to extravasate or escape from the microvessel, invade the surrounding tissue, and proliferate to form a metastatic tumor.

The GP Ibα inhibitor may be a peptide, an antibody, a small molecule, or a venom protein, as detailed above in section I(a). Modes of administration of the GP Ibα inhibitor were detailed above in section I(b). The various types of cancer whose spread may be reduced were detailed above in section I(c).

### DEFINITIONS

To facilitate understanding of the invention several terms are defined below.

Glycoprotein Ibα (GP Ibα) is the alpha subunit of the GP Ib, which also comprises a beta subunit. GP Ib non-covalently associates with GP IX to form the GP Ib-IX complex, which is an adhesion receptor in the cell membrane of platelets.

von Willebrand factor (vWF), the ligand of GP Ib-IX, is a large multimeric glycoprotein (i.e., more than 80 vWG monomers associate to form a multimer).

As various changes could be made in the above-described methods and compositions without departing from the scope of the invention, it is intended that all matter contained in the above description and the examples presented below, shall be interpreted as illustrative and not in a limiting sense.

### EXAMPLES

The following examples are further illustrative of the present invention.

### Examples 1-3

### Platelet Glycoprotein Ibα Supports Experimental Lung Metastasis

### Introduction

The following examples were designed to examine the role that platelet accumulation may play in tumor cell metastasis. That is, the adhesive properties of platelets and the elaboration of a fibrin matrix may provide a mechanism for circulating tumor cells to metastasize. The platelet-specific adhesion receptor glycoprotein (GP) Ib-IX is critical in hemostasis and thrombosis in that it initiates the formation of a platelet-rich thrombus by tethering the platelet to thromogenic surface. The role of GP Ib-IX in tumor metastasis was examined by inducing experimental metastasis in mice having GP Ib-IX deficiencies. Experimental metastasis refers to the injection of tumor cells directly into the circulation, e.g., via injection into the lateral tail vein, leading to major metastases in the lung.

### Experimental Protocols

Mice. Control C57BL/6J animals were obtained from The Jackson Laboratory (Bar Harbor, Me.). GP Ib-IX-deficient animals have been previously described and were generated by a gene targeting strategy of the mouse GP Ibα gene (GP1b) (Ware et al., 2000, Proc. Natl. Acad. Sci. USA 97:2803-2808). The platelet GP Ib-IX receptor complex is assembled from three distinct platelet-specific gene productions with mutations in any of the subunits producing the Bernard-Soulier syndrome (BSS). In the mouse model of GP Ibα-deficiency, the coding sequence for GP Ibα was deleted leading to a complete lack of detectable platelet GP-Ib-IX. The mouse BSS colony was backcrossed (10 generations, N10) with C57BL/6J mice purchased from The Jackson Laboratory. The breeding scheme involved stabilizing the mouse Y chromosome in generation one (N1) by using male C57BL/6J animals and choosing heterozygous male offspring for subsequent generations. The breeding of heterozygous GP1b^{+/-} animals to normal C57BL/6J animals in generation 10 led to GP1b^{+/-} progeny that were bred to each other, generating homozygous mice of the BSS phenotype (B6.129S7-GP Ib^{tm1}). For simplicity, these animals are referred to as GP1b^{-/-}. All GP1b^{-/-} mice used herein had been previously screened by flow cytometry to confirm the absence of a mouse GP Ib-IX complex.

Three additional mouse colonies expressing variants of the GP Ibα subunit have been previously described (Ware et al., 2000, supra; Kanaji et al., 2002, Blood 100:2102-2107; Kanaji et al., 2004, Blood 104:3161-3168). In brief, each colony was bred onto a mouse background devoid of murine GP Ibα alleles (GP1b^{-/-}) and backcrossed with control C57BL/6J animals for 10 generations to generate congenic animals in a strategy similar to that described above. However, in each case, animals were screened by flow cytometry at each generation to insure the presence of a transgenic product. One colony expresses a variant GP Ibα subunit where most of the extracytoplasmic sequence of GP Ibα has been replaced by an isolated domain of the interleukin-4 receptor fused to the transmembrane and cytoplasmic residues of GP Ibα (Kanaji et al., 2002, supra). Herein, these congenic animals are designated, IL-4R. Two additional colonies expressing transgenic products express either the full-length human GP Ibα sequence (designated, hTg^{WT}) or a six-residue truncation of the cytoplasmic tail (designated, hTg^{Y605X}) (Ware et al., 2000, supra; Kanaji et al., 2004, supra). All animal procedures were performed with institutional guidelines and approval.

Antibodies and Flow Cytometry. Whole blood was analyzed by flow cytometry (FACscan, Becton Dickinson, Franklin Lakes, N.J.) using a variety of phycoerythrin-labeled or fluorescein isothiocyanate (FITC)-conjugated antibodies. An anti-mouse CD41 (anti-GPIIb or αIIb) monoclonal antibody (Cat. No. 558040, BD Pharmingen, San Jose, Calif.) was used to identify the platelet population in whole blood. After identifying the platelet population, a gate was set in the flow cytometer to analyze fluorescence produced by a second labeling with a FITC-conjugated rat anti-mouse CD42b (anti-GP Ibα) monoclonal antibody to confirm the absence of mouse GP Ibα(Xia.G5, available from Emfret Analytics, Eibelstadt, Germany). For confirmation of human transgene expression, a FITC-conjugated mouse anti-human CD42b monoclonal antibody (Cat. No. 555472, BD Pharmingen) was used.

To evaluate the B16F10.1 cell-platelet interaction, whole blood was drawn from anesthetized mice via the retroorbital plexus by using heparinized capillary tubes. Platelet-rich plasma was removed after centrifugation (200×g for 5 min), and a platelet pellet was generated after another centrifugation (2,000×g for 5 min). Platelets were resuspended in modified Tyrode's buffer (140 mM NaCl, 2.7 mM KCl, 10 mM NaHCO₃, 0.42 mM Na₂HPO₄, 5 mM dextrose, 1 mM CaCl₂, and 10 mM Hepes, pH 7.4) and washed a second time after a similar centrifugation and resuspension in modified Tyrode's buffer. Platelet counts were determined, and tumor cells were added to generate a final ratio of 1:200 (tumor cell-to-platelets). The samples were kept at room temperature (RT) for ˜20 min, antibody was added (30-min RT incubation), and the mixture was diluted 3-fold with modified Tyrode's buffer before analysis by flow cytometry.

Cells. B16F10.1 murine melanoma cells were obtained from American Type Culture Collection. Cells were cultured in Dulbecco modified Eagle medium (DMEM) with 10% fetal bovine serum, 100 units/ml penicillin, 100 units/ml streptomycin, 2 mM glutamine, 10 mM Hepes, and 1 mM sodium pyruvate in the presence of 5% CO₂.

Experimental metastasis. B16F10.1 cells were supplied with fresh medium one day prior to their harvest for tail vein injection. Subconfluent cells (70-80%) were washed with Dulbecco's phosphate-buffered saline and detached by brief exposure to trypsin (0.25% trypsin, 0.2% EDTA) and washed twice with serum-free medium. Cells were resuspended in serum-free medium and kept on ice until injection. Viability was determined by trypan blue exclusion and was always more than 95%. Two hundred pl of tumor cell (1×0⁵ cells) suspension was injected to the lateral tail vein of mice using a 27-gauge needle.

Quantitation of surface pulmonary metastatic foci. Mice containing lung tumors were sacrificed on day 14 after tumor cell injection. The lungs were removed and rinsed in saline and weighed. Lungs were kept in Bouin's fixative for 24 hr before counting. Individual lobes were separated, and the number of surface-visible metastases was determined using a stereomicroscope (×2 magnification, Tritech Research, Los Angeles, Calif.). Statistical analysis was performed by using the Student's t test.

Ferric chloride-induced thrombosis. For ferric chloride (FeCl₃)-induced carotid artery injury, the carotid artery was exposed on anesthetized mice (2.5% isoflurane). A 4×10 mm strip of Whatman No. 1 filter paper was soaked with 10% FeCl₃ and placed on the exposed artery for 3 min. After removal of the filter paper, the exposed area was thoroughly rinsed with isotonic saline. Blood flow was monitored using a laser Doppler system (Trimflo, Vasamedics Inc., Eden Prairie, Minn.) connected to a BPM² blood perfusion monitor (Vasamedics) interfaced via an analogue to digital output with software from PowerLab System (AD Instruments Pty Ltd., Castle Hill, Australia).

### Example 1

### Platelet GP Ibα and Experimental Metastasis

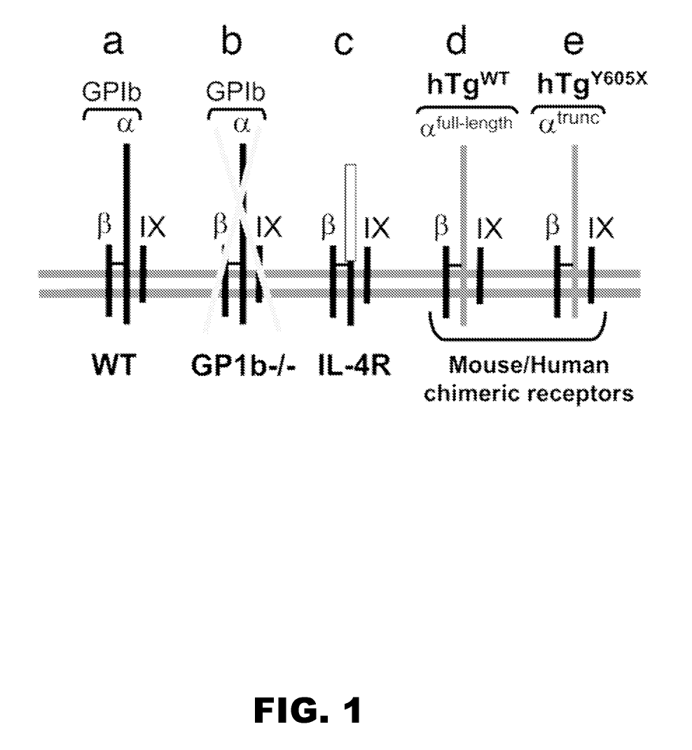

Two mouse models of platelet glycoprotein Ib-IX deficiency have been described (Ware et al., 2000, supra; Kanaji et al., 2002, supra). Briefly, a knockout of the platelet GP Ib subunit (GP1b^{-/-}) generates a murine model of the human Bernard-Soulier syndrome (BSS). These mice have a severe bleeding phenotype, macrothrombocytopenia, and no detectable GP Ib-IX receptor on their platelet surface. The second model was developed by partially rescuing the GP1b^{-/-} macrothrombocytopenic phenotype by transgenic expression of a variant GP Ib subunit (IL-4R). The variant subunit consists of an extracellular domain of the human IL-4 receptor fused to human GP Ib transmembrane and cytoplasmic domains. These mice retain a severe bleeding phenotype, owing to the absence of GP Ib extracytoplasmic domains but, as compared with GP1b^{-/-} mice, have an increased platelet count and a more normal distribution of platelet size in whole blood (Kanaji et al., 2002, supra). For the purpose of these examples, both mouse models have been backcrossed for 10 generations to C57BL/6J mice, generating congenic strains of each model (FIG. 1).

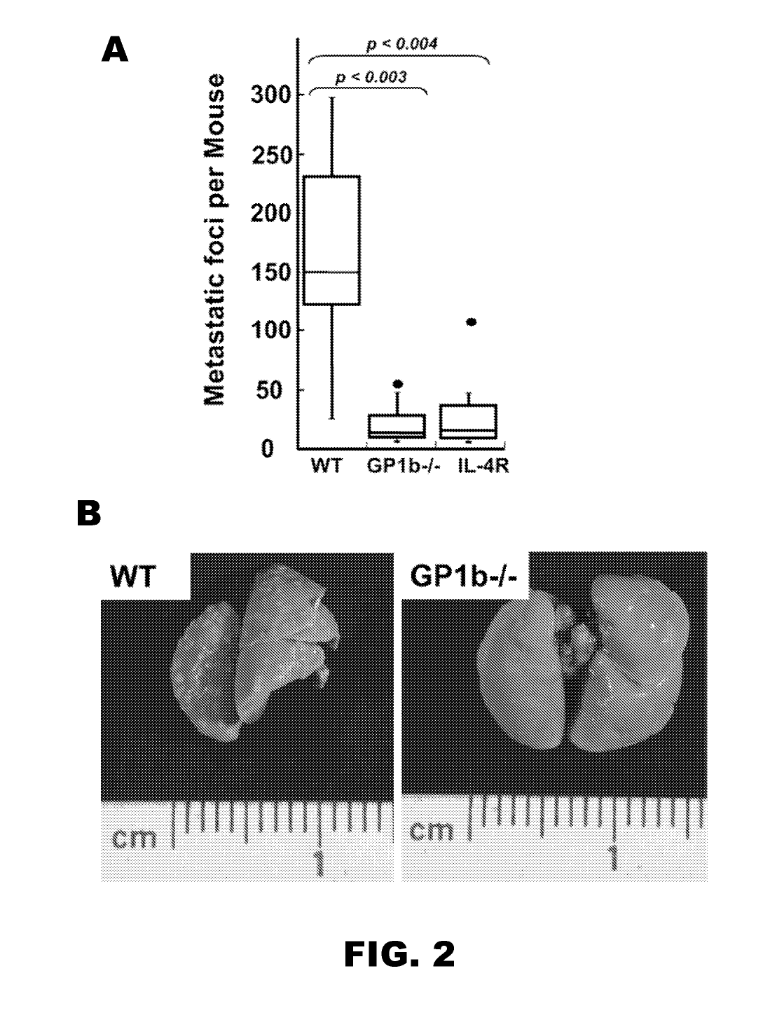

A syngeneic model of experimental metastasis was used to determine the physiologic relevance of platelet GP Ibα in tumorigenesis. Metastatic murine melanoma cells B16F10.1 (B16) were injected into the lateral tail vein of mice and 14 days later the extent of lung metastasis determined. Following euthanasia, lungs were dissected and the number of surface-visible lung tumors was determined. Results are shown following the injection of 1×10⁵ B16 cells in a series of age- and sex-matched control (C57BL/6J), congenic GPIb^{-/-}, and congenic IL4-R animals (FIG. 2). The average number of visible tumors in GPM^{-/-} was 19-fold less than that observed in control C57BI/6J animals. The median value for GPIb^{-/-} mice was 8, whereas the median value for control lungs was 150 (FIG. 2). The reduction in surface metastases was statistically significant with the p-value of 0.0001. Similar results were obtained in two independent experiments. The number of tumor foci was strongly dependent on the presence of GP Ib-IX, but the size and overall appearance of individual foci was indistinguishable between control C57BIL/6J and GPIb^{-/-} lungs.

As mentioned above, GPIb^{-/-} mice display macrothrombocytopenia with circulating platelet counts approximately one-third of a normal value. Thus, to determine the significance of the GPIb^{-/-} associated macrothrombocytopenia, experiments were performed using the congenic IL-4R mouse model, still devoid of extracytoplasmic GP Ibα functions but with an ameliorated macrothrombocytopenia. Visible lung tumors in IL-4R mice had a median value of 12, as compared to the median value of 150 tumors with C57BL/6J controls (FIG. 2). The reduced number of metastatic foci on IL-4R lungs was statistically significant with a P value of 0.004. No statistical difference was observed between GPIb^{-/-} and IL-4R lungs.

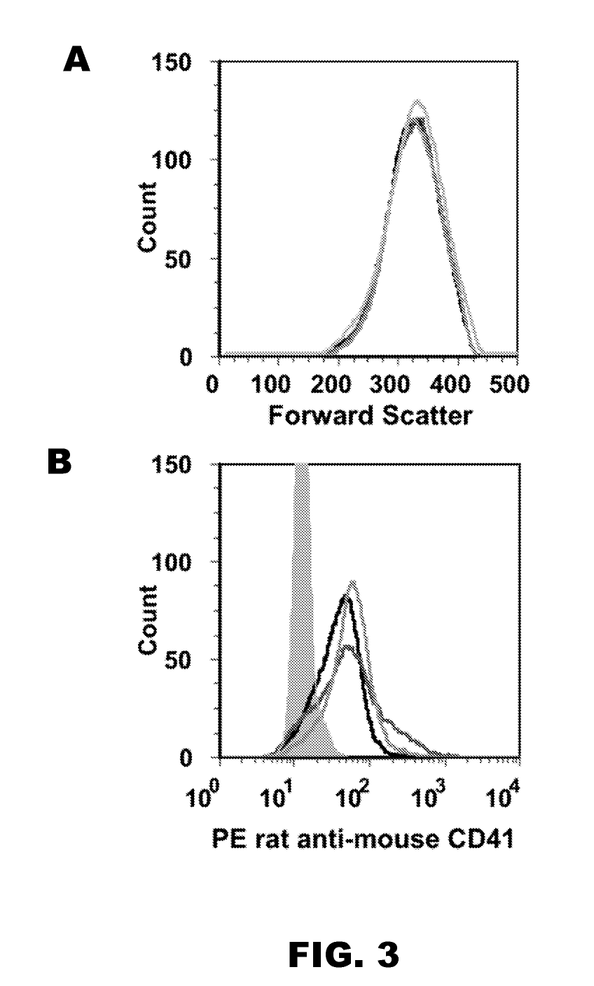

To further evaluate platelet/tumor cell interactions, B16 cells were mixed with washed platelets at a 1:200 ratio (i.e., B16:platelets). Flow cytometry analysis gating on tumor cells compared fluorescence in the presence of labeled platelets from C57BL/6J, GP1b^{-/-}, and IL-4R animals. No obvious fluorescent profile differences were seen among the three mouse strains (FIG. 3). Thus, it was concluded that under these experimental conditions, there is not a major role for platelet GP Ib-IX in a platelet-tumor cell interaction.

### Example 2

### Human GP Ibα and Experimental Thrombosis

Mice devoid of mouse GP Ibα, GPIb^{-/-}, but expressing a platelet-specific transgene encoding human GP Ibα, hTg^{WT} have been previously described (Ware et al., 2000, supra). HTg^{WT} mice have a rescued Bernard-Soulier phenotype as evidence by increased platelet count, a normal distribution of platelet size, and normal hemostasis. A similar mouse colony has also been generated that expresses a truncated form of the human GP Ibα transgene lacking 6-terminal residues that interact with the transduction protein, 14-3-3ζ (Kanaji et al., 2004, supra). These mice, hTg^{Y605X}, have been characterized for the relevance of these cytoplasmic residues in megakaryocyte maturation and proliferation. It was found that the cytoplasmic truncation had no effect on circulating platelet counts or hemostasis, as determined in a tail bleeding-time assay.

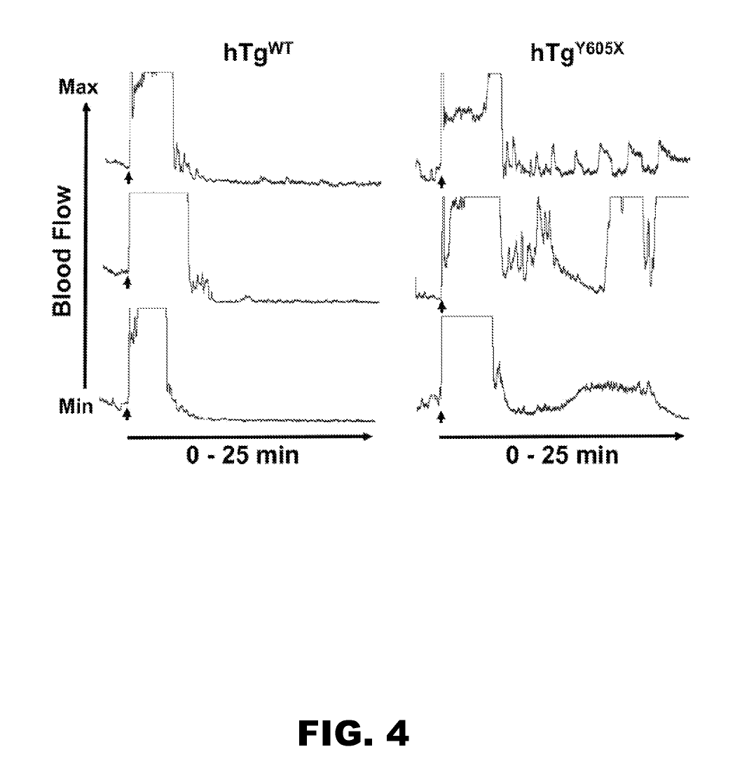

The relevance of theses cytoplasmic residues for thrombosis is presented in FIG. 4. In a model of ferric chloride-induced carotid artery injury, mice expressing hTg^{WT} were observed to have a rapid and stable reduction of blood flow, leading to an occlusion that persists for at least 25 min. In total, 10 hTg^{WT} animals were tested, with indistinguishable results from the three representative tracings presented in FIG. 4. This result supports the previous finding that human GP Ibα expressed on the surface of mouse GP Ib-deficient platelets does rescue the mouse BSS phenotype (Ware et al., 2000, supra). In contrast, mice expressing the cytoplasmic truncation, hTg^{Y605X}, had impaired thrombosis in this model. Blood flow reduction indicative of thrombus formation occurred, but in contrast to hTg^{WT}, there was evidence of fluctuating blood flow indicative of embolization and an inability to completely occlude the vessel (FIG. 4). Similar results were obtained from 10 different animals and three representative tracings are shown (FIG. 4). Thus, truncation of GP Ibα does impact thrombosis in this ferric chloride model and presumably relates to alterations in a GPIbα/14-3-3ζ signaling pathway.

### Example 3

### Human GP Ibα and Experimental Metastasis

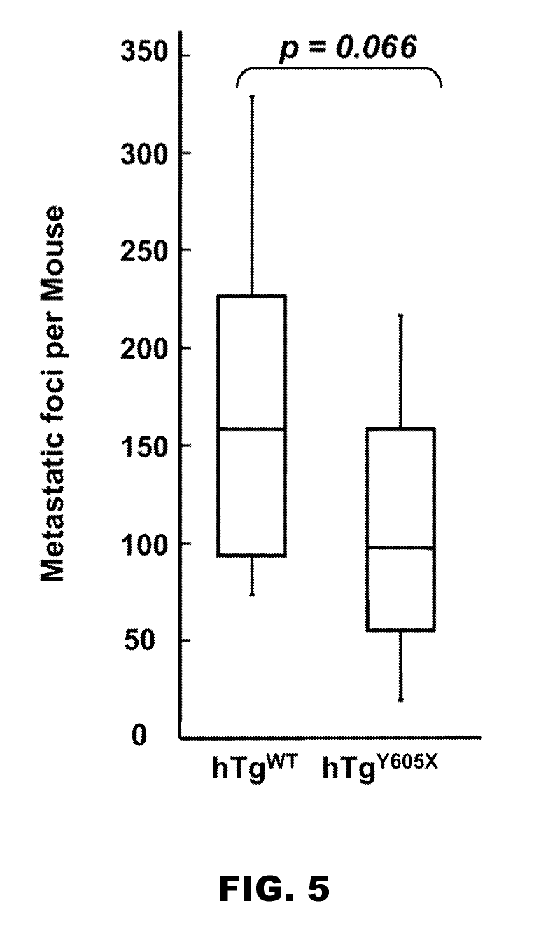

Having established both hTg^{WT} and hTg^{Y605X} animals as congenic strains, experiments were performed to determine whether human GP Ibα supports experimental metastasis and to determine whether the cytoplasmic interactions, such as GP Ibα/14-3-3ζ signaling pathways, might contribute to the process. B16 cells were injected in the tail veins of hTg^{WT} and hTg^{Y605W} animals. The expression of human GP Ibα in this model of mouse GP Ibα deficiency produced a significant number of visible tumors, median=157, thus confirming the ability of human GP Ibα to support tumor development (FIG. 5). Likewise, the six-residue truncation of GP Ibα did not significantly impact the relevance of GP Ibα to support metastasis (median value, =96). No statistical significance was observed between the two groups (P-value of 0.066). This result demonstrates that human GP Ibα supports metastasis and the GP Ibα/14-3-3ζ dependent-signaling pathways are not relevant to the formation of lung tumors in this model of experimental metastasis. These results indicate that GP Ib-IX can support experimental metastasis in platelets unable to from stable thrombi. In combination with results obtained with IL-4R mice, these results support the hypothesis that the extracellular domain of platelet GP Ibα supports experimental metastasis.

In conclusion, these examples demonstrate that the extracellular domain of the α-subunit of GP Ib is the structurally relevant component of the GP Ib-IX complex contributing to metastasis. The results support the hypothesis that platelet GP Ib-IX functions that support normal hemostasis or pathologic thrombosis also contribute to tumor malignancy.

What is claimed is:

1. A method for reducing tumor cell metastasis in a subject, the method comprising administering to the subject an antibody that inhibits glycoprotein Ibα, such that tumor cell metastasis is reduced.

2. The method of claim 1, wherein the antibody is chosen from a monoclonal antibody or fragment thereof, a single chain antibody, an Fv fragment, an Fd fragment, an Fab fragment, an Fab′ fragment, a camelid antibody or fragment thereof, a chimeric antibody or fragment thereof, and a humanized antibody or fragment thereof.

3. The method of claim 1, wherein the antibody binds to a GP Ibα binding domain of von Willebrand factor or binds to an extracellular domain of glycoprotein Ibα.

4. The method of claim 1, further comprising administering at least one chemotherapeutic agent chosen from an alkylating agent, an anti-metabolite, an anti-tumor antibiotic, an anti-cytoskeletal agent, a topoisomerase inhibitor, an anti-hormonal agent, a targeted therapeutic agent, and combinations thereof.

5. The method of claim 1, wherein the antibody is a single chain antibody.

6. The method of claim 5, wherein metastatic tumor cells are reduced in number and distribution.

7. The method of claim 5, further comprising administering at least one chemotherapeutic agent chosen from an alkylating agent, an anti-metabolite, an anti-tumor antibiotic, an anti-cytoskeletal agent, a topoisomerase inhibitor, an anti-hormonal agent, a targeted therapeutic agent, and combinations thereof.

8. The method of claim 7, wherein metastatic tumor cells are substantially eliminated.

9. A method for reducing tumor cell malignancy in a subject, the method comprising administering to the subject an antibody that inhibits glycoprotein Ibα, such that tumor cell malignancy is reduced.

10. The method of claim 9, wherein reduced tumor cell malignancy is manifested by a reduction or inhibition of cell proliferation, a reduction or inhibition of tumor cell invasiveness, a reduction or inhibition of tumor cell metastasis, or combinations thereof.

11. The method of claim 9, wherein the antibody is chosen from a monoclonal antibody or fragment thereof, a single chain antibody, an Fv fragment, an Fd fragment, an Fab fragment, an Fab′ fragment, a camelid antibody or fragment thereof, a chimeric antibody or fragment thereof, and a humanized antibody or fragment thereof.

12. The method of claim 9, wherein the antibody binds to a GP Ibα binding domain of von Willebrand factor or binds to an extracellular domain of glycoprotein Ibα.

13. The method of claim 9, further comprising administering at least one chemotherapeutic agent chosen from an alkylating agent, an anti-metabolite, an anti-tumor antibiotic, an anti-cytoskeletal agent, a topoisomerase inhibitor, an anti-hormonal agent, a targeted therapeutic agent, and combinations thereof.

14. The method of claim 9, wherein the antibody is a single chain antibody.

15. A method for inhibiting formation of a tumor cell embolism in a subject, the method comprising administering to the subject an antibody that inhibits glycoprotein Ibα, such that formation of the tumor cell embolism is inhibited.

16. The method of claim 15, wherein the antibody is chosen from a monoclonal antibody or fragment thereof, a single chain antibody, an Fv fragment, an Fd fragment, an Fab fragment, an Fab′ fragment, a camelid antibody or fragment thereof, a chimeric antibody or fragment thereof, and a humanized antibody or fragment thereof.

17. The method of claim 15, wherein the antibody binds to a GP Ibα binding domain of von Willebrand factor or binds to an extracellular domain of glycoprotein Ibα.

18. The method of claim 15, further comprising administering at least one chemotherapeutic agent chosen from an alkylating agent, an anti-metabolite, an anti-tumor antibiotic, an anti-cytoskeletal agent, a topoisomerase inhibitor, an anti-hormonal agent, a targeted therapeutic agent, and combinations thereof.

19. The method of claim 15, wherein the antibody is a single chain antibody.


SEQUENCE LISTING (11 sequences)

SEQ ID NO: 1 - protein, 320 aa, Homo sapiens
EDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEAD

SEQ ID NO: 2 - protein, 269 aa, Homo sapiens
RRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPD

SEQ ID NO: 3 - protein, 264 aa, Homo sapiens
RRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPP

SEQ ID NO: 4 - protein, 260 aa, Homo sapiens
RRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPE

SEQ ID NO: 5 - protein, 256 aa, Homo sapiens
RRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCD

SEQ ID NO: 6 - protein, 259 aa, Homo sapiens
QEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMV

SEQ ID NO: 7 - protein, 242 aa, Homo sapiens
TTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMV

SEQ ID NO: 8 - protein, 226 aa, Homo sapiens
YCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMV

SEQ ID NO: 9 - protein, 202 aa, Homo sapiens
YCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPD

SEQ ID NO: 10 - protein, 197 aa, Homo sapiens
YCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPP

SEQ ID NO: 11 - protein, 193 aa, Homo sapiens
YCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPE